(19) United States
(12) Patent Application Publication
(10) Pub. No.: US 2018/0015291 A1
(43) Pub. Date: Jan. 18, 2018

(54) IMPLANTABLE MEDICAL DEVICE WITH ANTENNA FOR WIRELESS COMMUNICATION

(71) Applicant: BIOTRONIK SE & Co. KG, Berlin (DE)

(72) Inventors: Christian Moss, Berlin (DE); Christian Gothe, Berlin (DE); Thomas Finnberg, Zuerich (CH)

(21) Appl. No.: 15/637,563

(22) Filed: Jun. 29, 2017

(30) Foreign Application Priority Data
Jul. 13, 2016 (EP) 16179171.0

(51) Int. Cl. A61N 1/378; A61N 1/372; A61N 1/39

(52) U.S. Cl. CPC A61N 1/3787; A61N 1/37205; A61N 1/39; A61N 1/37211

(57) ABSTRACT

An implantable medical device having a hollow housing and a connector housing connected (e.g., rigidly) together, each containing electrical components. The components contained in the hollow housing include a receiver for wireless communication. The components contained in the connector housing include an antenna electrically connected to the receiver and configured to receive electromagnetic waves having a frequency greater than 100 MHz. The receiver/antenna allows the receipt of data in the MICS frequency band. The electrical components arranged in the connector housing additionally include a resonant circuit having a coil and a capacitor which is inductively coupled to the antenna. The resonant circuit is matched to magnetic alternating fields in a frequency range between 5 kHz and 50 MHz, preferably having a resonance frequency between 5 kHz and 50 MHz. The resonant circuit is suitable for data communication in the near field of the implantable medical device via magnetic alternating fields.

### CROSS-REFERENCE TO RELATED APPLICATIONS

This patent application claims the benefit of and priority to co-pending European Patent Application No. EP 16179171.0, filed on Jul. 13, 2016 in the European Patent Office, which is hereby incorporated by reference in its entirety.

### Field of the Invention

The present invention relates to an implantable medical device, for example, an implantable therapy device, which has a hollow housing and a connector housing, with the connector housing being connected to the hollow housing and with both the hollow housing and the connector housing containing electrical components. The electrical components contained in the hollow housing comprise a receiver for wireless communication, and the components contained in the connector housing comprise an antenna, which is electrically connected to the receiver in the hollow housing.

### Description of the Related Art

Implantable medical devices of this type can be therapy devices such as stimulation devices. Such stimulation devices are, for example, cardiac pacemakers, defibrillators, or also neurostimulators. Other implantable active medical devices are monitor devices, for example, for detecting and recording physiological parameters. In many cases, these implantable medical devices have a connector housing, which has connectors for the connection of electrical leads, which, for example, can be stimulation electrode leads or also sensor leads. The connector housing is often embodied as a plastics (cast) part, which is connected to a hollow housing, which comprises further components of the implantable medical device, such as a battery, evaluation electronics, and/or the like. The hollow housing is typically made of a biocompatible metal. Besides the components already mentioned, a telemetry transmitter and/or receiver can also be provided in the hollow housing so as to be able to wirelessly communicate uni- or bi-directionally with the implantable medical device. This is of interest, in particular, in conjunction with telemedicine, as is known per se, in which case an implantable medical device detects and stores physiological parameters and transmits these telemetrically to an external device. Data of this type can be, for example, an intracardial electrocardiogram or the like. In addition, data can also be transmitted to the implantable medical device via a telemetry arrangement of this type provided by the telemetry transmitter and receiver. The transmitter and/or receiver used for this purpose typically operates in a frequency range between 400 MHz and 460 MHz (MICS/MEDS or MedRadio) and in further frequency bands approved for implant communication.

Besides data communication in the MICS band, programmable implantable medical devices often also allow near-field communication, in which case, for example, an external programming device communicates with the implantable medical device via a magnetic alternating field (H-field). This form of data communication is particularly energy efficient.

The present invention is directed toward overcoming one or more of the above-mentioned problems.

An object of the present invention is to create an implantable medical device which allows efficient wireless communication.

### BRIEF SUMMARY OF THE INVENTION

At least this object is achieved in accordance with the present invention by an implantable medical device which has a hollow housing and a connector housing. The connector housing is connected, in particular rigidly connected, to the hollow housing. Both the hollow housing and the connector housing contain electrical components, of which the electrical components contained in the hollow housing comprise at least one receiver for wireless communication, and the components contained in the connector housing comprise at least one antenna which is configured to receive electromagnetic waves with a frequency of more than 100 MHz and which is electrically connected to the receiver. A receiver of this type which is connected to an antenna of this type by way of example allows the receipt of data in the MICS/MEDS and MedRadio frequency band. In accordance with the present invention, the electrical components arranged in the connector housing additionally comprise a resonant circuit which has a coil and a capacitor and which is inductively coupled to the antenna. The resonant circuit is matched to magnetic alternating fields in a frequency range between 5 kHz and 50 MHz and, accordingly, preferably has a resonance frequency between 5 kHz and 50 MHz. The resonant circuit is thus suitable for near-field data communication by means of the magnetic alternating fields.

The resonant circuit for data communication by means of magnetic alternating fields is thus preferably fully galvanically isolated from the electrical components arranged in the hollow housing.

The present invention makes it possible to arrange the coil for data transmission outside the hollow housing (specifically in the connector housing of the implant). The shielding effect of the hollow housing therefore does not come into play, and in addition the interfering influence by the implant electronics is negligible. An individual coil now no longer serves as receiver, but rather an inductively coupled system formed of a coil, which serves as an antenna and is electrically connected to the receiver or transceiver, and a resonant circuit, wherein the resonant circuit is of the highest possible quality. The combined structure can be integrated in the connector housing.

In contrast to known implantable medical devices, the resonant circuit does not need to be directly connected to a receiver for data communication by means of magnetic alternating field. Rather, the inventors have found that the resonant circuit can be inductively coupled to the antenna, likewise arranged in the connector housing, so that magnetic alternating fields exciting the resonant circuit can be coupled into the antenna of the implantable medical device designed for a much higher frequency range and can thus be fed via this antenna to the receiver of the implantable medical device.

An advantage of this solution is that, for example, only one bushing needs to be provided between the connector housing and hollow housing for connection of the antenna to the receiver, since the resonant circuit does not need to be directly galvanically connected to the receiver (or a second receiver) in the hollow housing.

At the same time, the disadvantages that are present if the resonant circuit for data communication by means of magnetic alternating field is accommodated in the hollow housing are also avoided. Since the hollow housing is typically a metal housing, magnetic alternating fields infiltrating the metal housing are heavily attenuated by this metal housing (although not completely shielded). This is not the case with a connector housing of the implantable medical device consisting typically of plastic, for example, epoxy resin, or another non-conductive material. The coil for data transmission is arranged in a titanium housing in the case of the implants available today. This is a simple system formed of two coils. In the case of conventional implantable medical devices with a coil integrated in a titanium housing, the magnetic field inside the hollow housing is attenuated by the hollow housing, whereby the extent of said magnetic field is reduced. In addition, in the case of conventional implantable medical devices, interfering radiation is encountered in the communication coil due to the rest of the electronics of the implant.

If a resonant circuit for optimizing the range is additionally used in conventional implantable medical devices, the quality of said resonant circuit is very low. This is due to the eddy current losses, which, in particular, result from the proximity to the metal of the hollow housing in the surroundings of the coil. These disadvantages are avoided by the implantable medical device according to the present invention.

A resonant circuit for data communication by means of magnetic alternating field arranged in the connector housing (and not in the hollow housing) additionally offers the advantage that the data communication can occur at higher frequencies, for example into the region of 50 MHz. This allows smaller component parts, in particular small coils. Frequencies of this type cannot be used if the coil for data communication by means of magnetic alternating field is arranged in a metal hollow housing, because frequencies in the range above 50 MHz are too heavily damped by the metal housing and higher frequencies are practically completely shielded.

With the aid of the present invention, the range in the case of data transmission with the aid of two coils inductively coupled to one another can be increased.

Magnetic sensors are used conventionally when waking up implants. Here, however, communication problems occur already in the case of patients having a high body mass index (BMI). Due to the increased range of the method according to the present invention, it is also logically possible to use the inductive coupling in order to wake up implants. In the case of the inductive coupling according to the present invention, the waking-up can be implemented in that the signal is passively rectified and is then fed to a threshold value decision-maker. The inductively transmitted signal generates an alternating current in the coils inductively coupled to one another, which alternating current can be rectified and compared to a threshold value, so that a switch-on signal is generated in the case of a sufficiently large amplitude of the rectified signal. The implantable medical device can contain a rectifier and a comparator for this purpose, which are electrically connected to the coils inductively coupled to one another.

If, in accordance with a preferred variant of the present invention, the terminating resistor of the antenna is additionally performance-matched to the ohmic resistor of the resonant circuit, the quality of the resonant circuit, which is to be expressed by the quality factor Q, is not adversely affected by the antenna.

In order to attain a performance matching of this type, the terminating resistor R_{L} is preferably dimensioned so that it satisfies the following equation:

$${R}_{L} = {{R}_{1} ( \frac{{N}_{2}}{{N}_{1}} )}^{2} ,$$

wherein N₁ is the number of turns which the coil of the resonant circuit has and N₂ is the number of turns which the antenna has, and R₁ is the ohmic resistor that acts in the resonant circuit.

The antenna is preferably formed by the coil, more specifically, in particular by a coil having a single turn. Accordingly, N₂=1.

The coil of the resonant circuit is preferably a coil having a number of turns N₁.

The coil of the resonant circuit is preferably oriented at least approximately concentrically with and/or parallel to the coil forming the antenna so as to thus obtain an optimal magnetic coupling of the two coils.

The capacitance of the resonant circuit is preferably formed by a capacitor. Alternatively, however, the capacitance can also be formed by an inherent capacitance of the coil itself.

Pulses of more than 10 μs length are preferably used for the excitation of the resonant circuit—i.e., for the data communication by means of magnetic alternating field—in order to ensure that the resonant circuit with its high quality has sufficient time to start resonating.

The frequencies used for the data communication by means of magnetic alternating field are 125 kHz, 13.56 MHz, and 37.5 MHz, for example. At greater frequencies (for example, from 2 MHz to 37.5 MHz), the coil of the resonant circuit simplifies to a few turns. Communication through a metal hollow housing would be ruled out at the higher of the aforementioned frequencies.

For data communication at higher frequencies between, for example, 2 MHz and 40 MHz, the resonant circuit can be formed by a coil with inherent resonance.

A metal that is a good conductor is preferably necessary as material for the resonant circuit and, in particular, coil thereof in order to achieve the best possible quality. Depending on the number of turns of the coil, however, the influence of the conductivity of the metal is negligible. A wire made of silver, followed by gold, followed by titanium, followed by high-grade steel, and followed by nitinol, is particularly suitable as material for the coil.

In accordance with an advantageous variant, the resonant circuit is formed by an inherently resonant coil. This can be a helix coil. By way of example, a coil with an inherent resonance at 39.7 MHz is suitable. This can have a factor of quality of Q=277 at this frequency. By way of example, the coil has 65 turns made of a wire 50 μm thick, which is wound at short distance so that the coil has a diameter of 10 mm. The coil then has a total length of approximately 4 mm. As a result of minor optimization, the inherent resonance can be adapted, for example, to 37.5 MHz. A frequency of 37.5 MHz is specified by way of example by REG70.03 for implants having an extremely low energy demand.

The hollow housing is preferably a metal housing made of a biocompatible metal, such as, for example, stainless steel or titanium. An advantage of a metal housing of this type is that the electrical components inside the hollow housing are shielded against electromagnetic radiation acting from outside. It is therefore also advantageous if the connector housing is formed substantially by a non-conductive material, such as for example, plastic, since the components arranged in the connector housing, such as the antenna and resonant circuit, are not shielded from electromagnetic or magnetic alternating fields.

In addition, the connector housing preferably has connectors for the connection of electrical leads, for example, stimulation or sensing electrode leads.

Further electrical components, in particular such as a battery, are preferably arranged in the hollow housing.

The receiver arranged in the hollow housing is preferably formed as a transmitter and receiver, i.e., as a transceiver, which can receive and transmit signals in the 3 to 4-figure megahertz range, and which in addition can receive signals in the kilohertz range to 2-figure megahertz range.

In accordance with the present invention, a method for wireless data communication between an external device and an implantable medical device by means of magnetic alternating fields is also proposed, in which method data is inductively transmitted from the external device to a resonant circuit of the implantable medical device, which is in turn coupled inductively to an antenna coil, so that signals received by the resonant circuit are coupled inductively into the antenna coil and are forwarded via said coil to a receiver or transmitter/receiver electrically connected to the antenna coil.

The data communication by means of magnetic alternating field preferably occurs at a frequency between 2 MHz and 50 MHz.

The method is preferably carried out with an implantable medical device of the type described here.

Further embodiments, features, aspects, objects, advantages, and possible applications of the present invention could be learned from the following description, in combination with the Figures, and the appended claims.

### BRIEF DESCRIPTION OF THE DRAWINGS

The above and other aspects, features and advantages of at least one embodiment of the present invention will be more apparent from the following more particular description thereof, presented in conjunction with the following drawings, wherein:

FIG. 1 shows an implantable medical device according to the present invention;

FIG. 2 shows an implantable medical device according to the prior art;

FIG. 3 shows some electrical components of an implantable medical device according to the present invention;

FIG. 4 shows a basic illustration of near-field data communication with the implantable medical device according to the present invention via a magnetic alternating field;

FIG. 5 shows a schematic illustration of the coupling of a resonant circuit and an antenna of the implantable medical device according to the present invention via a magnetic alternating field;

FIG. 6 shows a schematic illustration of the near-field coupling of an external device, such as a programming device, with the implantable medical device according to the present invention via a magnetic alternating field;

FIGS. 7A-B show an example of an inherently resonant helix coil which can be used as resonant circuit for the implantable medical device according to the present invention; and

FIG. 8 shows an illustration of the impedance curve of the coil illustrated in FIGS. 7A-B.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

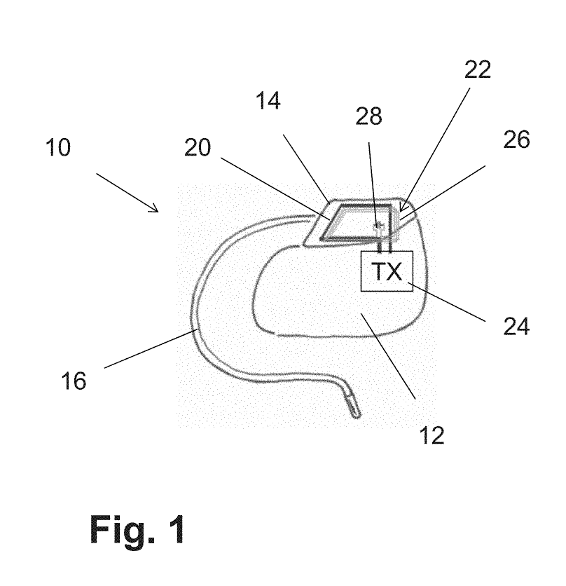

The implantable medical device 10 illustrated in FIG. 1 has a hollow housing 12, which is connected to a connector housing 14. A stimulation electrode lead 16 is connected to the connector housing 14.

An antenna 20 and a resonant circuit 22 are arranged in the connector housing 14. The antenna 20 is galvanically connected to a receiver 24, which is arranged inside the hollow housing 12. The resonant circuit 22 is coupled to the antenna 20 via a magnetic alternating field.

The resonant circuit 22 has a coil 26 and a capacitor 28, which forms a capacitance of the resonant circuit 22.

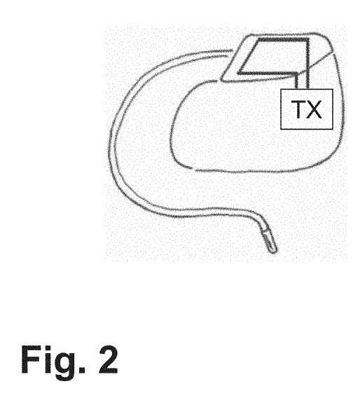

FIG. 2 shows an implantable medical device according to the prior art, which has a transmitter/receiver (transceiver) TX in a hollow housing, which is connected to an antenna, which is arranged in the connector housing of the implantable medical device. As can be seen from a comparison of FIGS. 1 and 2, the implantable medical device 10 according to the present invention additionally has the resonant circuit 22 arranged in the connector housing 14 for inductive data transmission.

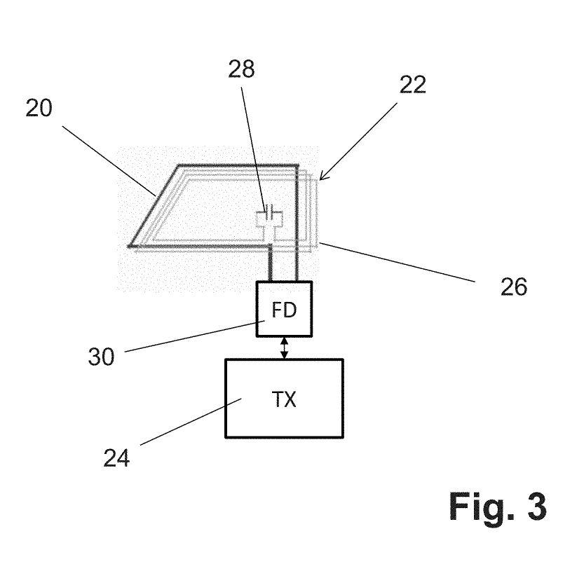

FIG. 3 shows a more detailed illustration of some of the electronic components of the implantable medical device 10. As can be inferred from FIG. 3, the transmitter/receiver 24 is connected to the antenna 20 via a bushing 30, which is formed by a coil having a single turn.

In addition, FIG. 3 shows the resonant circuit 22, which has a coil 26 having a number of turns N₁ and a capacitance formed by a capacitor 28. As shown in FIG. 3, the coil forming the antenna 20 and the coil 26 of the resonant circuit 22 can be arranged exactly one above the other.

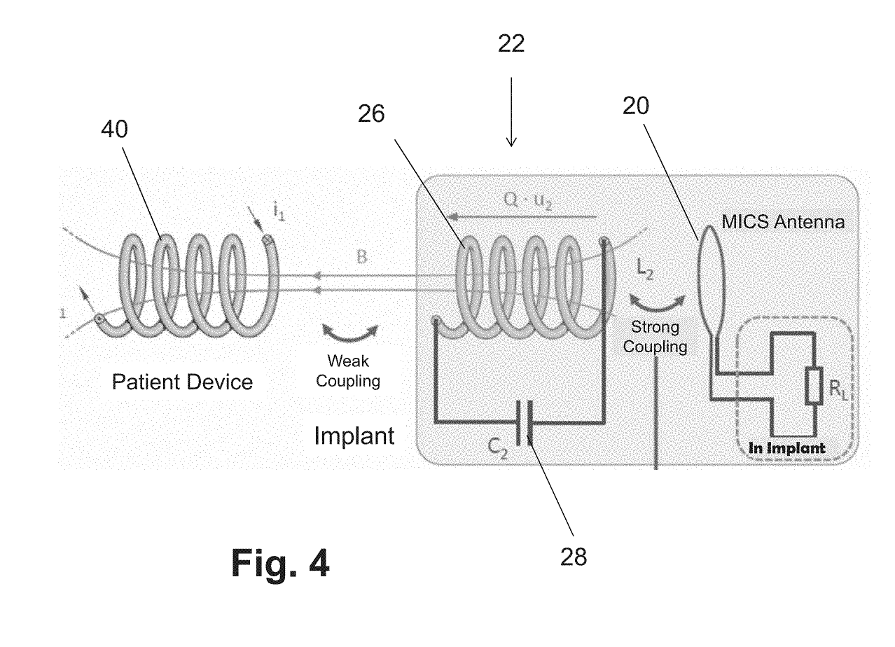

FIG. 4 serves to explain an inductive data transmission from a patient device to the implantable medical device with its resonant circuit 22, which has the coil 26 and the capacitor 28. As can be inferred from FIG. 4, the inductive coupling between the coil 40 of the external device and the coil 26 of the resonant circuit 22 is weak.

A strong inductive coupling on the other hand exists within the connector housing 14 between the coil 26 of the resonant circuit 22 and the coil forming the antenna 20.

FIG. 4 also indicates that the coil forming the antenna is terminated with a terminating resistor R_{L} arranged in the hollow housing (i.e., in the implant).

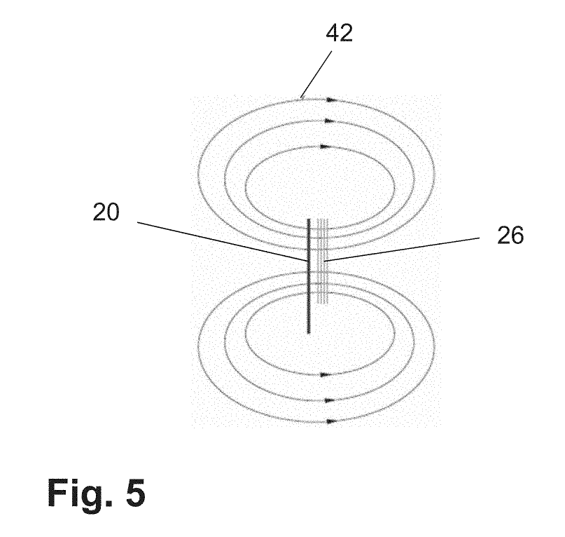

FIG. 5 illustrates the strong inductive coupling between the antenna 20 formed as a coil and the coil 26 of the resonant circuit 22. The magnetic field lines 42 of the magnetic field which acts between the antenna 20 and the coil 26 of the resonant circuit 22 are indicated.

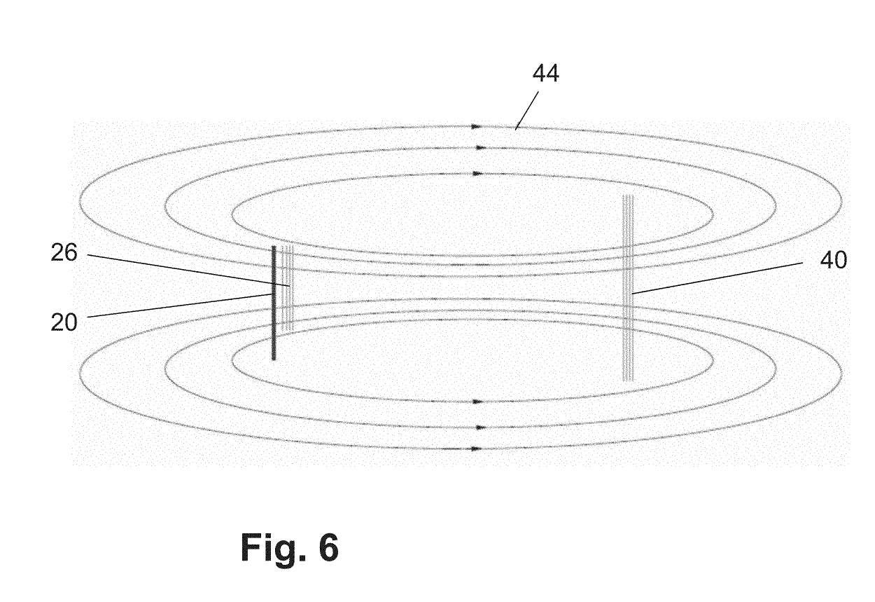

FIG. 6 illustrates the comparatively weaker inductive coupling between a coil 40 of an external device and the coil 26 of the resonant circuit 22 in the connector housing 14 of the implantable medical device 10. Magnetic field lines 44 are again also indicated in FIG. 6 and represent the magnetic field via which the coil 40 of the external device is coupled inductively to the coil 26 of the resonant circuit 22.

The solution presented in FIGS. 1, 3 and 4 is based on the use of the MICS antenna 20, which is already provided in the connector housing 14 of the active implant 10. This antenna 20 is guided via a bushing 30 towards the inside of the hollow housing 12. The antenna is connected there to the MICS transceiver 24 via a matching network.

The MICS antenna 20 is formed by a coil having just one turn; see FIGS. 1 and 3. In addition to this coil of the antenna 20, a resonant circuit 22 is arranged in the connector housing 14, the resonance frequency of said resonant circuit being equal to the carrier frequency of the inductive data transmission and said resonant circuit having a further coil 26 and a capacitor 28; see FIG. 1. This resonant circuit 22 is coupled inductively to the MICS antenna 20; see FIGS. 4 and 5.

By optimal configuration of the MICS antenna 20 and resonant circuit 22—in particular, by varying the coupling factor and number of turns—it is possible to achieve a matching between the resonant circuit 22 and the terminating resistor R_{L} of the MICS antenna 20. In contrast to conventional methods, in the case of this approach the quality of the resonant circuit 22 is not reduced by insertion into an electrical circuit, whereby a greater range can be attained. FIG. 6 shows the inductive coupling between a patient device outside the body and an active implant comprising the described coupled coils.

In order to attain the desired power matching and not adversely affect the quality Q of the resonant circuit 22, the antenna 20 is preferably terminated with an ohmic terminating resistor R_{L} satisfying the following equation:

$${R}_{1} = {{R}_{L} ( \frac{{N}_{1}}{{N}_{2}} )}^{2} ,$$

R₁ is here the ohmic resistor which acts in the resonant circuit. N₁ is the number of turns of the coil of the resonant circuit, and N₂ is the number of turns of the coil forming the antenna.

One possibility for the specific conversion has already been shown in FIGS. 1 and 3. Here, both coils are arranged exactly one above the other. In another embodiment, the resonant circuit 22, by way of example, could occupy a much smaller area than the MICS antenna 20. The resonance frequency is dependent on the inductance of the coil determined by the size (geometry) of the coil and the number of turns of the coil and by the electrical properties, in particular the intrinsic capacitance, and the other electrical components coupled to the coil.

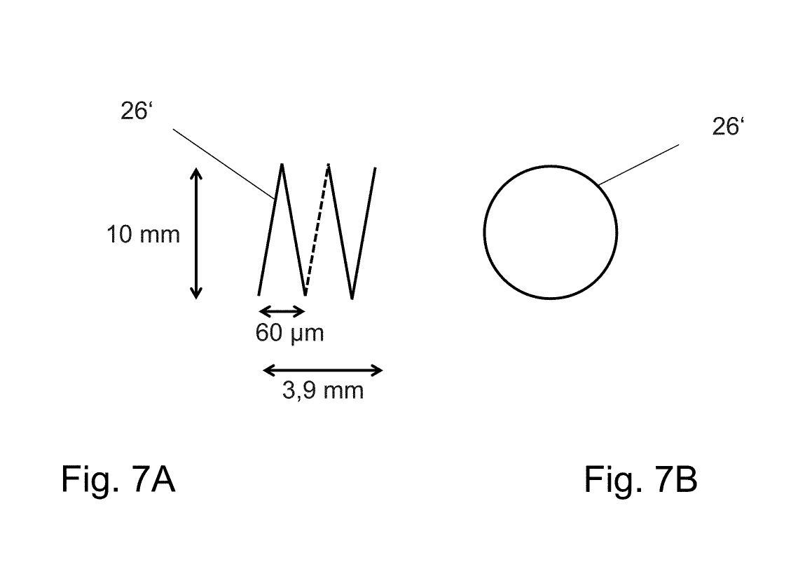

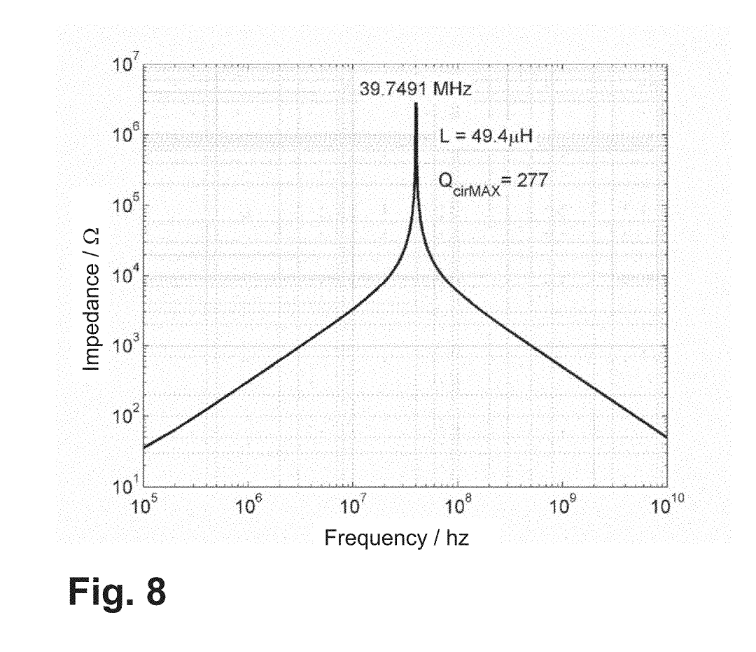

In accordance with an alternative embodiment, an inherent resonance frequency of the coil 26′ of the resonant circuit 20 can be used for the data transmission; see FIGS. 7A-B and 8. It is thus possible to dispense with additional component parts and the connector housing 14. An example of such an inherently resonant coil 26′ is shown in FIGS. 7A-B. FIG. 8 shows the associated impedance curve of the inherently resonant coil 26′ illustrated in FIGS. 7A-B. As can be inferred from this impedance curve, the coil 26′ has an inherent resonance at 39.7 MHz. If an inherently resonant coil 26′ of this type is used for the resonant circuit 22, the resonant circuit 22 does not need an additional capacitor, and instead the capacitor of the inherently resonant coil 26′ is sufficient as (sole) capacitor of the resonant circuit 26.

As can be inferred from FIGS. 7A-B, a suitable inherently resonant coil 26′ has a diameter of 10 mm and a length of approximately 3.9 mm and has 65 turns (N₁=65). The coil is wound as a helix and has a total length of approximately 4 mm. The inherently resonant coil 26′ is formed by a wire made of a metal that is a good conductor having a wire diameter of 50 μm. The pitch of the coil turns is 60 μm, i.e., the adjacently arranged wires of the individual coil turns are arranged at a distance of approximately 10 μm from one another. FIG. 7A schematically shows a side view of a suitable inherently resonant coil 26′ of this type, and FIG. 7B shows the corresponding plan view. As can be inferred from FIG. 7B, the inherently resonant coil 26′ is circular in the plan view.

A metal that is a good conductor, for example, a wire made of silver, followed by gold, followed by titanium, followed by high-grade steel and followed by nitinol, is particularly suitable as material for the coil 26′.

The coil 26′ has an inherent resonance at 39.7 MHz and at this frequency has a factor of quality of Q=277. As a result of minor optimisation, the inherent resonance can be changed to 37.5 MHz.

The presented inductive telemetry with large range and simultaneously reduced interfering radiation within the implant also enables an energy-saving activation of the implant due to the increased range. For this purpose, the implantable medical device can contain a rectifier and a comparator, which are electrically connected to the coils coupled inductively to one another. The signal transmitted inductively generates an alternating current in the coils coupled inductively to one another, which alternating current can be rectified and compared to a threshold value so that a switch-on signal can be generated in the case of a sufficiently large amplitude of the rectified signal.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

10 implantable medical device

12 hollow housing

14 connector housing

16 stimulation electrode device

20 antenna

22 resonant circuit

24 transmitter/receiver (transceiver)

26, 26 coil of the resonant circuit 22

28 capacitor of the resonant circuit 22

30 bushing

40 coil of an external device

42, 44 magnetic field lines

We claim:

1. An implantable medical device comprising:
a hollow housing and a connector housing, which is connected to the hollow housing, wherein both the hollow housing and the connector housing contain electrical components, of which the electrical components contained in the hollow housing comprise a receiver for wireless communication and the components contained in the connector housing comprise an antenna, which is configured to receive electromagnetic waves having a frequency of more than 100 MHz and which is electrically connected to the receiver, wherein the electrical components contained in the connector housing additionally comprise a resonant circuit, which has a coil and a capacitor and which is coupled inductively to the antenna, wherein the resonant circuit is configured to respond to magnetic alternating fields in a frequency range between 5 kHz and 50 MHz.

2. The implantable medical device according to claim 1, wherein the receiver in the hollow housing and the antenna in the connector housing are electrically connected to one another via a bushing.

3. The implantable medical device according to claim 1, wherein the antenna is formed by a coil having a single turn.

4. The implantable medical device according to claim 1, wherein the coil of the resonant circuit is a coil having a plurality of turns.

5. The implantable medical device according to claim 3, wherein the coil of the resonant circuit is oriented at least approximately concentrically with and/or parallel to the coil forming the antenna.

6. The implantable medical device according to claim 1, wherein the antenna and the resonant circuit are power-matched to one another in such a way that the quality of the resonant circuit is not adversely affected.

7. The implantable medical device according to claim 6, wherein the antenna is terminated with a terminating resistor (R_{L}) which is dimensioned as follows:
$${R}_{L} = {{R}_{1} ( \frac{{N}_{2}}{{N}_{1}} )}^{2} ,$$
wherein N₁ is the number of turns that the coil of the resonant circuit has and N₂ is the number of turns that the antenna has, and R₁ is the ohmic resistor that acts in the resonant circuit.

8. The implantable medical device according to claim 1, wherein the resonant circuit has a resonance frequency between 5 kHz and 50 MHz.

9. The implantable medical device according to claim 1, wherein the capacitance of the resonant circuit is formed by a capacitor.

10. The implantable medical device according to claim 1, wherein the resonant circuit is formed by an inherently resonant coil having an inherent resonance frequency between 2 MHz and 50 MHz.

11. The implantable medical device according to claim 1, wherein the resonant circuit has a quality Q which is greater than 200.

12. A method for wireless data communication between an external device and an implantable medical device by means of magnetic alternating fields, in which method data is transmitted inductively from the external device to a resonant circuit of the implantable medical device, which is in turn coupled inductively to an antenna coil so that signals received by the resonant circuit are coupled inductively into the antenna coil and are forwarded via this coil to a receiver or transmitter/receiver electrically connected to the antenna coil.

13. The method according to claim 12, wherein the data communication is performed by means of magnetic alternating field at a frequency between 2 MHz and 50 MHz.

14. A method for wireless data communication between an external device and an implantable medical device by means of magnetic alternating fields, in which method data is transmitted inductively from the external device to a resonant circuit of the implantable medical device, which is in turn coupled inductively to an antenna coil so that signals received by the resonant circuit are coupled inductively into the antenna coil and are forwarded via this coil to a receiver or transmitter/receiver electrically connected to the antenna coil, wherein the method is carried out with an implantable medical device according to claim 1.
